(19) United States
(12) Patent Application Publication
(10) Pub. No.: US 2026/0069798 A1
(43) Pub. Date: Mar. 12, 2026

(54) METERED DOSE INHALER STORAGE CONTAINER

(71) Applicant: MDI PLUS LIMITED, Athy, County Kildare (IE)

(72) Inventors: Mohamed Emam Abdelmobdy ABDELRAHIM, Giza Governorate (EG); David LITTLE, Stradbally, County Laois (IE)

(73) Assignee: MDI PLUS LIMITED, Athy, County Kildare (IE)

(21) Appl. No.: 19/101,839

(22) PCT Filed: Aug. 10, 2023
(86) PCT No.: PCT/EP2023/072201

§ 371 Date: Feb. 6, 2025

(30) Foreign Application Priority Data
Aug. 10, 2022 (EG) EG/P/2022/1164

(51) Int. Cl. A61M 15/00

(52) U.S. Cl. CPC A61M 15/0021; A61M 2205/0205; A61M 2205/0238; A61M 2209/06

(57) ABSTRACT

A metered dose inhaler storage container comprises a packaging carton which has openings at opposite ends of the packaging carton comprising an inlet opening at one end of the packaging carton for reception of a mouthpiece of a metered dose inhaler and an associated outlet opening at an opposite end of the packaging carton for engagement by a mouth of a user to inhale medication discharged from the metered dose inhaler through the packaging carton which forms a spacer for the metered dose inhaler. The metered dose inhaler is supplied in the packaging carton and stored within the packaging carton when not in use.

The packaging carton forms both a storage container and a spacer for the metered dose inhaler.

### INTRODUCTION

This invention relates to a metered dose inhaler storage container, and in particular to a metered dose inhaler packaging carton.

### BACKGROUND TO THE INVENTION

Metered-dose inhalers (MDIs) are the most commonly used devices for aerosol delivery to the airways. However, most patients, especially the elderly and pediatrics do not use it correctly by committing a poor inhalation technique. In other words, a slow inspiration (around 30 L/min) should be done by the patient while actuating the MDI (Hand-breath coordination). Moreover, when using MDIs, at least 50-80% of the drug deposits in the mouth and the throat with only a very small amount (10-15%) of the drug reaching the airways. Hence, placing a reservoir device (Spacer) between the MDI and the patient's mouth gives more time for drug inhalation as it gives more volume and space for the propellant to evaporate. In other words, these add-on spacer devices allow large particles emitted by the MDI deposit within it rather than being deposited in the mouth and throat. Also, the extra distance travelled by the aerosol within the add-on spacer devices increases the dose of fine particles, less than 5 μm, which is the optimal particle size that can reach the deep smaller airways. Various types of spacer device are available, and they may be very large, collapsible and/or disposable. However, these spacer devices are relatively expensive, and many are bulky and inconvenient for a patient to carry around and have available when using their metered dose inhaler.

The present invention is directed towards addressing these problems.

### SUMMARY OF THE INVENTION

According to the invention, there is provided a metered dose inhaler storage container which can form a spacer for the metered dose inhaler, the storage container having a pair of spaced-apart openings in a side wall of the storage container comprising an inlet opening for reception of an outlet of the metered dose inhaler and an associated outlet opening for engagement by a mouth of a user to inhale medication discharged from the metered dose inhaler and delivered through the storage container.

The side wall of the storage container may be formed in any suitable way and typically will comprise a plurality of interconnected panels. The outlet opening is for engagement by a mouth, with or without an additional mouthpiece, of the user.

In a particularly preferred embodiment, the storage container comprises a packaging carton or box for reception and storage of the associated metered dose inhaler prior to use and in between uses of the metered dose inhaler by a patient.

In one embodiment of the invention, a removable closure is provided at each opening.

In another embodiment, the removable closure is a flap mounted across the opening.

In another embodiment, the flap is a tear-away flap.

In another embodiment, the flap is mounted in the opening.

In another embodiment, a periphery of the flap connects to a periphery of the opening by a perforated seam, or a punched or perforated line of weakness.

In another embodiment, the flap is hingedly mounted at a side of the opening for movement between a closed position extending across the opening and an open position away from the opening.

In another embodiment, the removable closure is a peel-away tab.

In another embodiment, the tab is a peel and re-seal tab. The tab may be a board flap or tuck-in panel or label tab.

In another embodiment, the inlet opening is shaped for complementary reception and engagement of the mouthpiece of the metered dose inhaler within the inlet opening. The inlet opening can be any suitable shape as different MDIs will have mouthpieces of different shape. It is desirable to have a snug fit between the mouthpiece and the inlet opening.

In another embodiment, the preceding claims, wherein the storage container is impregnated with an anti-microbial agent.

In another embodiment, the anti-microbial agent comprises anti-microbial metal ions.

In another embodiment, at least an interior surface of the storage container is coated with an anti-static material to minimise drug retention.

In another embodiment, the storage container has a coating of plastics material or a water based dispersion coating.

The invention provides a metered dose inhaler storage container which can form a spacer for the metered dose inhaler, the container having an access opening for insertion and removal of the metered dose inhaler, a closure mounted at the access opening, the closure being operable to open and close the access opening, the storage container having a pair of spaced-apart openings in a side wall of the storage container comprising an inlet opening for reception of an outlet of the metered dose inhaler and an associated outlet opening for engagement by a mouth of a user to inhale medication discharged from the metered dose inhaler and delivered through the storage container.

In another aspect, the invention provides the storage container in combination with an associated metered dose inhaler which houses within the storage container.

In another embodiment, the inlet opening has a periphery shaped to receive and engage an exterior of the mouthpiece of the metered dose inhaler.

In another embodiment, the outlet opening has a periphery shaped to receive an additional patient mouthpiece.

In another aspect, the invention provides a blank for forming the metered dose inhaler storage container. The blank may be in any carton style or design.

In another embodiment, the blank comprises a front panel, rear panel and side panels arranged side by side in a row joined along side edges, the front panel being connected at one side edge to a first side panel by a first fold line and on the other side edge to one side edge of a second side panel by a second fold line, an opposite side edge of the second side panel connecting to a first side edge of the rear panel by a third fold line, a connector flap projecting outwardly of a second side edge of the rear panel by a fourth fold line, an adhesive strip on a face of the connector flap for engagement with the first side panel, a top panel hingedly connected to a top edge of the rear panel and a bottom closure flap connected to a bottom edge of the rear panel, a bottom panel connected to a bottom edge of the front panel and a top closure flap connected to a top edge of the front panel.

In another embodiment, the panels are of rectangular shape.

### BRIEF DESCRIPTION OF THE DRAWINGS

The invention will be more clearly understood by the following description of some embodiments thereof, given by way of example only, with reference to the accompanying drawings, in which:

FIG. 1 is a perspective view of a metered dose inhaler storage container according to the invention forming a packaging carton for a metered dose inhaler;

FIG. 2 is a perspective view of the metered dose inhaler packaging carton according to the invention;

FIG. 3 is a perspective view of the packaging carton, shown in use;

FIG. 4 is a perspective view of the metered dose inhaler packaging carton, shown in another position of use;

FIG. 5 is another perspective view of the packaging carton shown in another position of use;

FIG. 7 is a perspective view of the packaging carton, shown in use as a spacer for a metered dose inhaler;

FIG. 8 is a perspective view showing the metered dose inhaler packaging carton in use as a spacer;

FIG. 9 is a plan view of a blank for forming the metered dose inhaler packaging carton of the invention.

FIG. 10 is a perspective view of the metered dose inhaler packaging carton;

FIG. 11 is a perspective view of another metered dose inhaler packaging carton according to a second embodiment of the invention;

FIG. 12 is a perspective view of a further metered dose inhaler packaging carton according to a third embodiment of the invention;

FIG. 13 is a perspective view of a metered dose inhaler packaging carton according to another embodiment of the invention;

FIG. 14 is a perspective view of a metered dose inhaler packaging carton according to a further embodiment of the invention;

FIG. 15 is a perspective view of a metered dose inhaler packaging carton according to another embodiment of the invention;

FIG. 16 is a perspective view of a further metered dose inhaler packaging carton according to another embodiment of the invention;

FIG. 17 is a perspective view of a metered dose inhaler packaging carton according to another embodiment of the invention; and

FIG. 18 is a perspective view of a metered dose inhaler packaging carton according to a further embodiment of the invention.

### DETAILED DESCRIPTION OF THE PREFERRED EMBODIMENTS

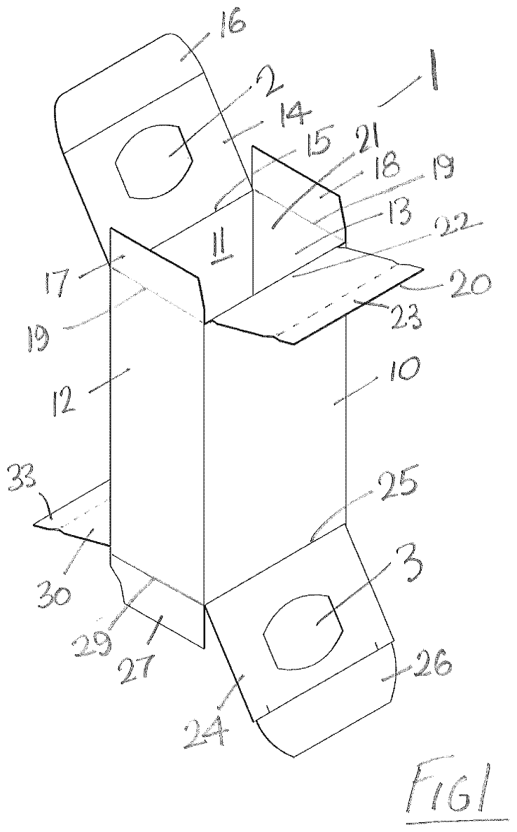

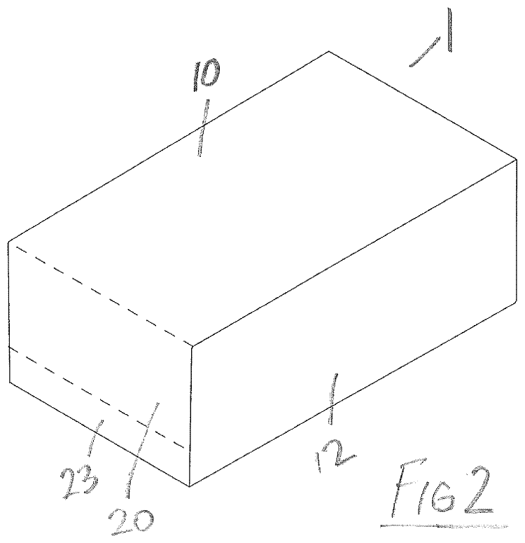

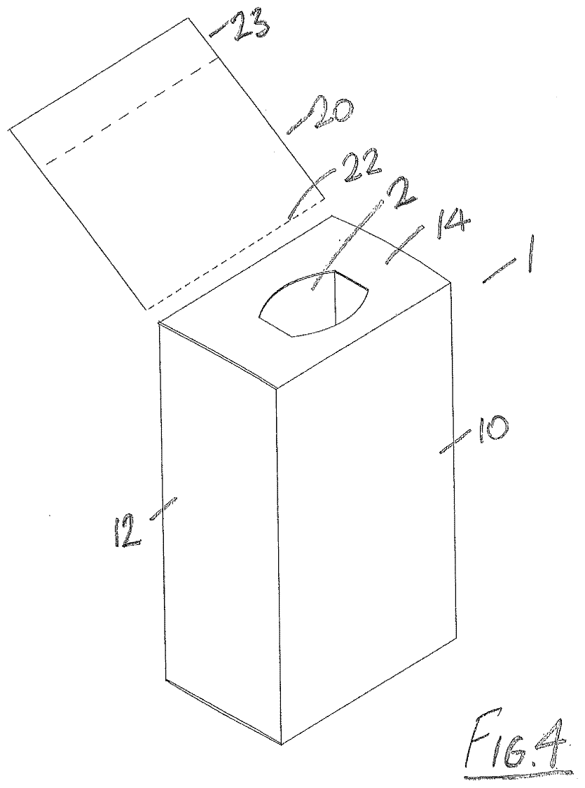

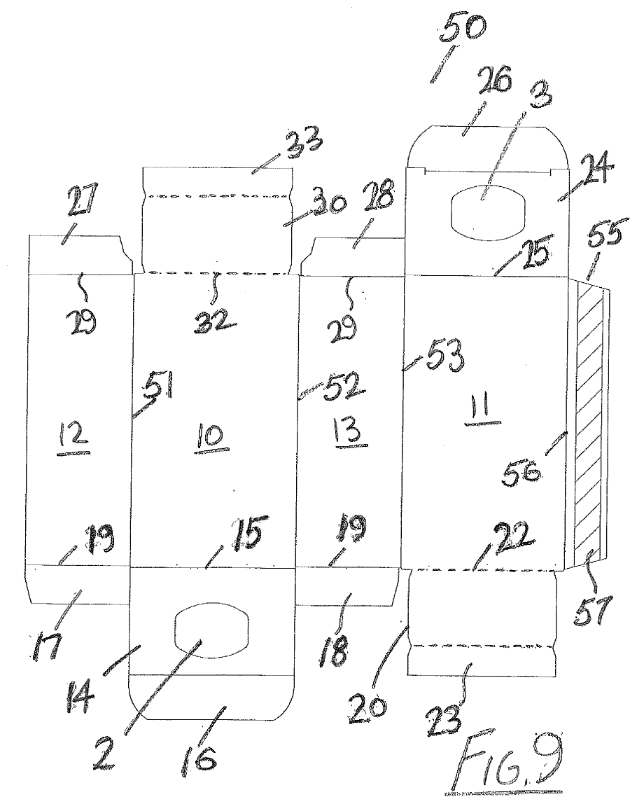

Referring to the drawings, and initially to FIG. 1 to FIG. 10 thereof, there is illustrated a metered dose inhaler storage container according to the invention forming a packaging carton, indicated generally by the reference numeral 1. The packaging carton 1 has openings 2, 3 at opposite ends of the packaging carton 1 comprising an inlet opening 2 at one end of the packaging carton 1 for reception of a mouthpiece 4 of a metered dose inhaler 5 and an associated outlet opening 3 at an opposite end of the packaging carton 1 for engagement by a mouth of a user 6 (see FIG. 8) to inhale medication discharged from the metered dose inhaler 5 through the packaging carton 1 which forms a spacer for the metered dose inhaler 5.

The packaging carton 1 has a rectangular front panel 10 and a rectangular rear panel 11 interconnected by rectangular side panels 12, 13 extending therebetween. A top panel 14 connects by a hinge 15 to an upper end of the rear panel 11. A top locking flap 16 at a free end of the top panel 14 is engagable with an upper end of the front panel 10 to releasably secure the top panel 14 in a closed position. Associated top side flaps 17, 18 are hingedly connected to the side panels 12, 13 by fold lines 19 at upper ends of the side panels 12, 13 and fold inwardly under the top panel 14 to retain the locking flap 16 in a closed position. The inlet opening 2 is provided in the top panel 14. The top panel 14 forms a closure at an access opening 21 at a top of the packaging carton 1 for insertion and removal of the metered dose inhaler 5.

A top tamper evident closure flap 20 initially is folded over and overlies an outside face of the top panel 14, closing the inlet opening 2. The closure flap 20 is connected to a top edge of the front panel 10 by a perforated hinge line 22 and has a free outer end 23 adhesively secured to an outer face of the top panel 14. This closes the inlet opening 2 of the packaging carton 1 initially but the closure flap 20 can be detached from the packaging carton 1 to expose the inlet opening 2 for use.

In similar fashion, at a bottom of the packaging carton 1 a bottom panel 24 connects by a hinge 25 to a lower end of the front panel 10. A bottom locking flap 26 at a free end of the bottom panel 24 is engagable with a lower end of the rear panel 11 to releasably secure the bottom panel 24 in a closed position. Associated bottom side flaps 27, 28 are hingedly connected to the side panels 12, 13 by fold lines 29 at lower ends of the side panels 12, 13 fold inwardly above the bottom panel 24 to retain the bottom locking flap 26 in a closed position. The outlet opening 3 is provided in the bottom panel 24. The bottom panel 24 could be glued closed, in a skillet end for example.

A bottom tamper evident closure flap 30 initially is folded over an outside face of the bottom panel 24, closing the outlet opening 3. The bottom closure flap 30 is connected to a bottom edge of the rear panel 11 by a perforated hinge line 32 and has a free outer end 33 adhesively secured to an outer face of the bottom panel 24. This closes the outlet opening 3 of the packaging carton 1 initially but the bottom closure flap 30 can be detached from the packaging carton 1 to expose the outlet opening 3 for use.

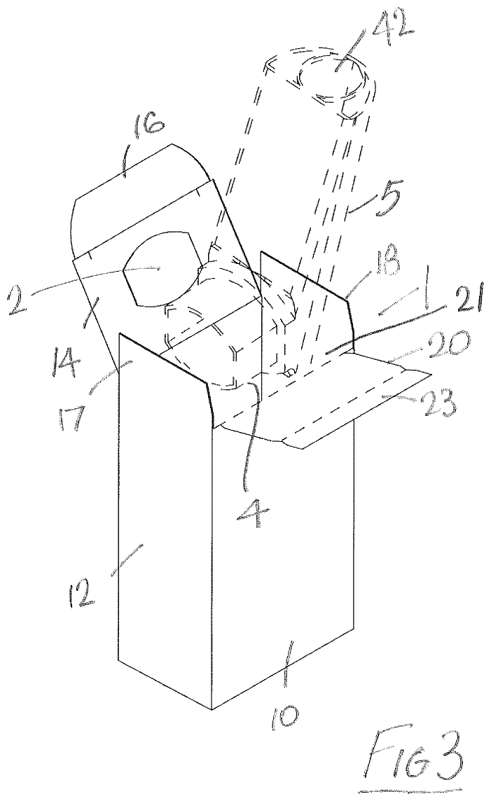

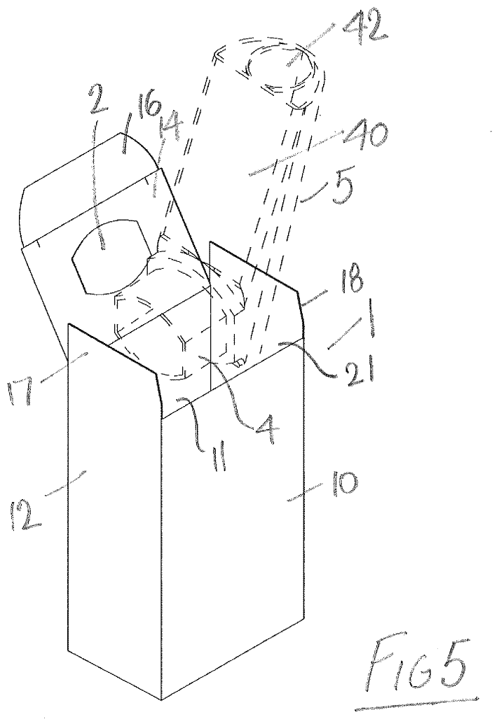

FIG. 3 shows the top closure flap 20 and top panel 14 in open positions to allow removal of the metered dose inhaler 5 stored within the packaging carton 1. FIG. 5 is a similar view to FIG. 3, showing the packaging carton 1 with the top closure flap 20 (and bottom closure flap 30) removed thus converting the packaging carton 1 into a spacer for use with the metered dose inhaler 5.

The metered dose inhaler 5 has a housing 40 containing medication. A metered dose of the medication can be dispensed through the mouthpiece 4 at an outlet of the housing 40 when an actuating button 42 on the housing 40 is depressed. Typically, the metered dose inhaler 5 contains a plurality of metered doses of the medication.

FIG. 6 shows the packaging carton 1 converted into a spacer, an interior of the packaging carton 1 forming a chamber with the inlet opening 2 and outlet opening 3 in a top panel 14 and bottom panel 24 at opposite ends of the chamber.

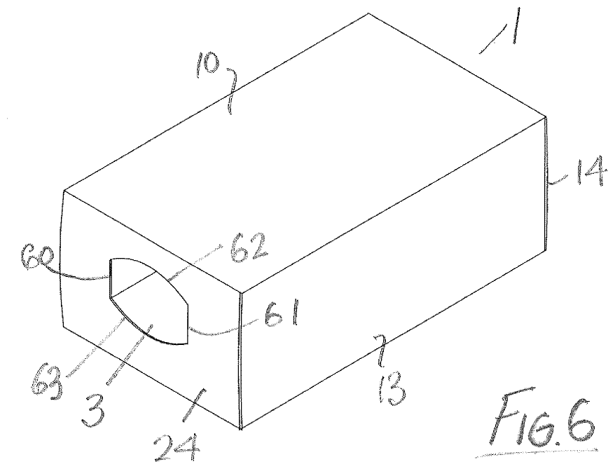

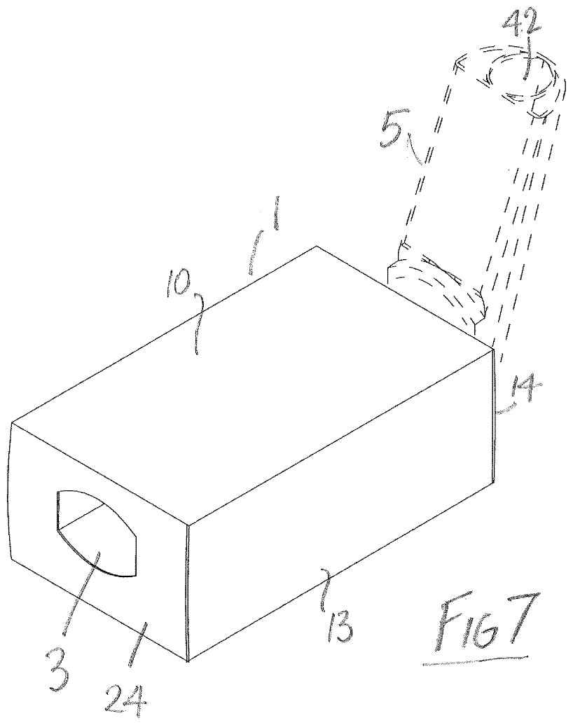

FIG. 7 shows the mouthpiece 4 of the metered dose inhaler 5 inserted into the inlet opening 2, within which it is a close fit. As shown in FIG. 8, the user 6 can then position their mouth at the outlet opening 3 at an opposite end of the packaging carton 1 (now acting as a spacer) and by manually operating the actuating button 42 deliver a metered dose of medication from the metred dose inhaler 5, through the packaging carton 1 and out through the outlet opening 3 and into the mouth of the user 6.

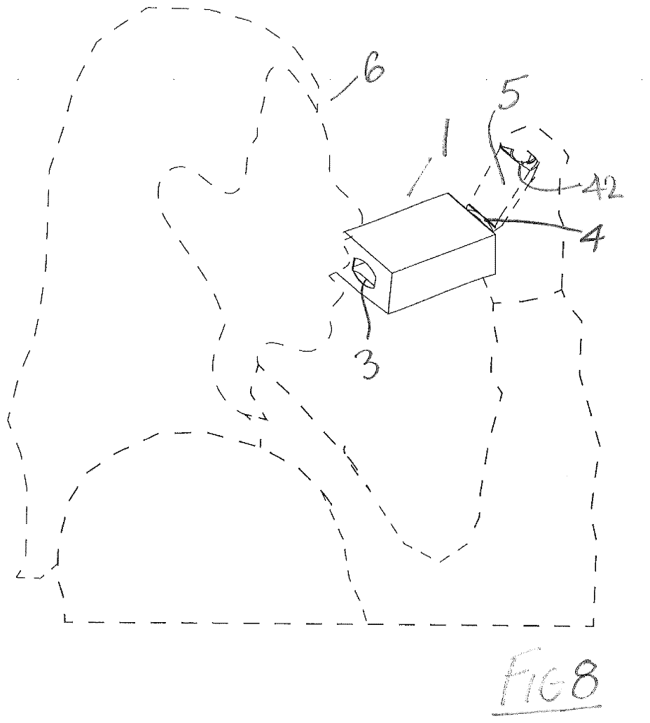

FIG. 9 shows a blank 50 for forming the packaging carton 1. The front panel 10, rear panel 11 and side panels 12, 13 are arranged side by side in a row. The front panel 10 is connected on one side by a fold line 51 with one side panel 12 and on the other side by a fold line 52 with one side of the other side panel 13. An opposite side of the side panel 13 connects by fold line 53 with one side of the rear panel 11. A connector flap 55 projects outwardly of the other side of the rear panel 11 to which it is connected by a fold line 56. An adhesive strip 57 on a face of the connector flap 55 is engagable with an inside face of the side flap 12 at the opposite side of the blank 50 to form the packaging carton 1.

The packaging carton 1 comprises a card material that may be impregnated with an anti-microbial agent such as anti-microbial metal ions. Optionally, at least an inner surface of the packaging carton 1 is coated with an anti-static material to minimise drug retention. The packaging carton 1 may also be provided with a coating of plastics material, a bio/water based coating or a plastics coating or film laminate.

In this case, the openings 2, 3 are similar so that the opening 2, 3 at either end of the packaging carton 1 can form the inlet opening 2 for engagement by the mouthpiece 4 of the metered dose inhaler 5 and the outlet opening 3 for engagement by the mouth of the user 6. It is envisaged, however, that different shaped openings could be provided at opposite ends of the packaging carton 1.

Referring in particular to FIG. 6, it will be noted that each opening 2, 3 has parallel straight end edges 60, 61 interconnected by curved side edges 62, 63. This shape of opening 2, 3 corresponds to the exterior profile of the mouthpiece 4 of the metered dose inhaler 5. A periphery of the opening 2, 3 snugly engages with an external surface of the mouthpiece 4 intermediate an inner end and an outer end of the mouthpiece 4, essentially forming a seal between the periphery of the opening 2, 3 and the mouthpiece 4 to prevent leakage of medication when using the packaging carton 1 as a spacer.

In use, a fully charged metered dose inhaler 5 is supplied within an associated packaging carton 1 and is secured within the fully closed packaging carton 1. When required for use, the top closure flap 20 and bottom closure flap 30 are torn away and discarded. Upon opening the top panel 14 or the bottom panel 24, the metered dose inhaler 5 is removed from the packaging carton 1. The top or bottom panel 14, 24 is then closed again to convert the packaging carton 1 into a spacer for the metered dose inhaler 5, as shown in FIG. 6. The metered dose inhaler 5 is then mounted at the inlet opening 2, engaging the mouthpiece 4 of the metered dose inhaler 5 within the inlet opening 2, as shown in FIG. 7. Holding the metered dose inhaler 5 in engagement with the inlet opening 2 (FIG. 8), the user 6 then positions the outlet opening 3 at their mouth and operates the actuating button 42 to deliver a metered dose of medication from the metered dose inhaler 5, through the packaging carton 1, which is now acting as a spacer, and through the outlet 3 into the mouth of the user 6. After use, the metered dose inhaler 5 is disengaged from the inlet opening 2 and inserted into the packaging carton 1 within which it can conveniently be stored between uses.

It will be appreciated that the invention provides a simple, compact, cheap and sustainable way of providing a spacer device for use with a metered dose inhaler. The packaging carton of the invention can conveniently and quickly be converted into a spacer when required for use with the metered drug inhaler and when the metered drug inhaler is empty, the packaging carton can be readily easily disposed in an environmentally friendly way.

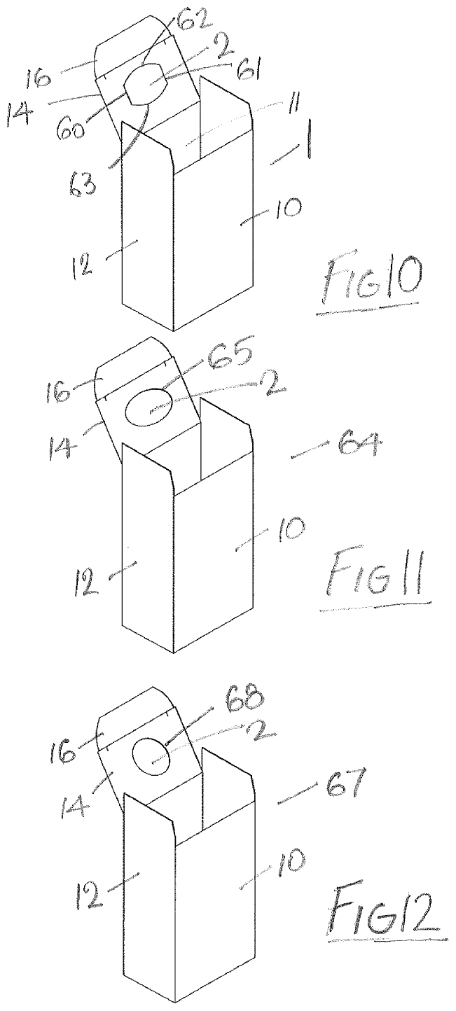

Referring now to FIG. 11, there is shown a metered dose inhaler packaging carton according to a second embodiment of the invention, indicated generally by the reference numeral 64. Parts similar to those described previously are assigned the same reference numerals. In this case, an inlet opening 2 (and outlet opening 3) having an oval periphery 65 is provided on the top panel 14 and bottom panel 24.

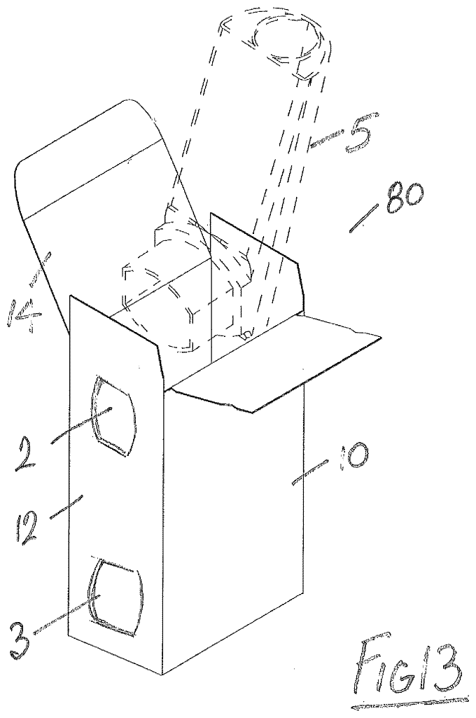

Referring now to FIG. 12, there is shown another metered dose inhaler packaging carton according to a third embodiment of the invention, indicated generally by the reference numeral 67. Parts similar to those described previously are assigned the same reference numerals. In this case, the inlet and outlet openings 2, 3 have a circular periphery 68.

Any suitable material may be used to form the storage container such as card or plastics such as PET (polyethylene terephthalate) or acetate. Stiff card is a particularly useful and cheap material for forming a carton or box for housing the metered dose inhaler and forming the spacer device when required. In an alternative arrangement, the storage container might be formed by an injection moulded plastics box with a hinged lid. This would provide a more robust storage container, if required.

It will be appreciated that the inlet opening 2 and the outlet opening 3 may be provided in various panels 10, 11, 12, 13, 14, 24 and in various arrangements on these panels 10, 11, 12, 13, 14, 24 on opposed panels 10, 11, 12, 13, 14, 24 or adjacent panels 10, 11, 12, 13, 14, 24 or even on the same panel 10, 11, 12, 13, 14, 24.

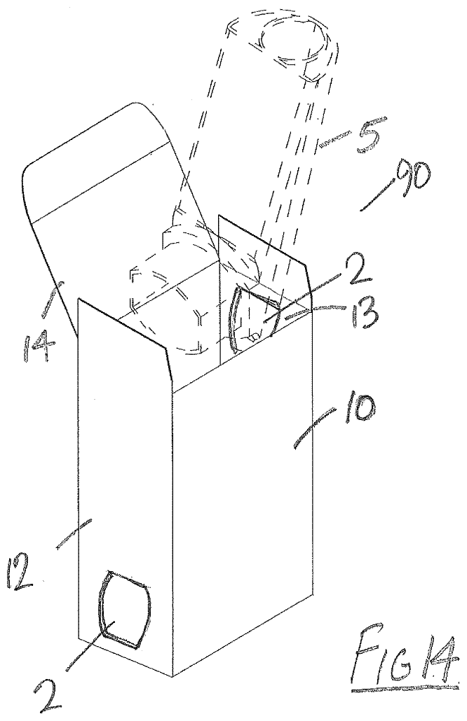

Referring now to FIG. 13, there is shown another metered dose inhaler packaging carton according to a fourth embodiment of the invention, indicated generally by the reference numeral 80. Parts similar to those described previously are assigned the same reference numerals. In this case, the inlet opening 2 and the outlet opening 3 are provided spaced-apart on the same side panel 12.

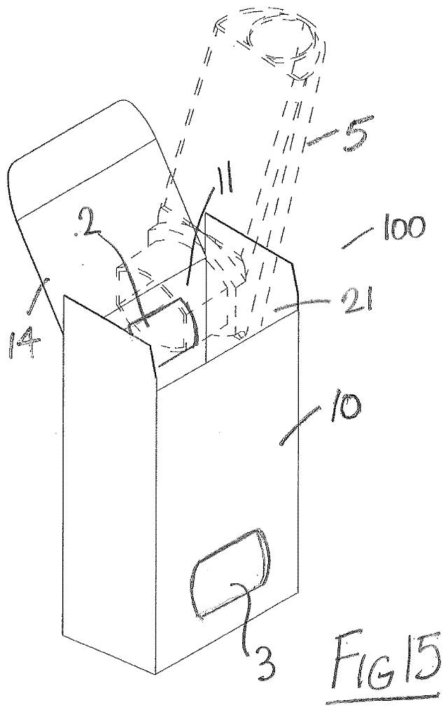

Referring now to FIG. 14, there is shown a further metered dose inhaler packaging carton according to a fifth embodiment of the invention, indicated generally by the reference numeral 90. Parts similar to those described previously are assigned the same reference numerals. In this case, the inlet opening 2 and the outlet opening 3 are provided spaced-apart at a top of the side panel 13 and at a bottom end of the side panel 12 respectively, at an opposite side of the metered dose inhaler packaging carton 90.

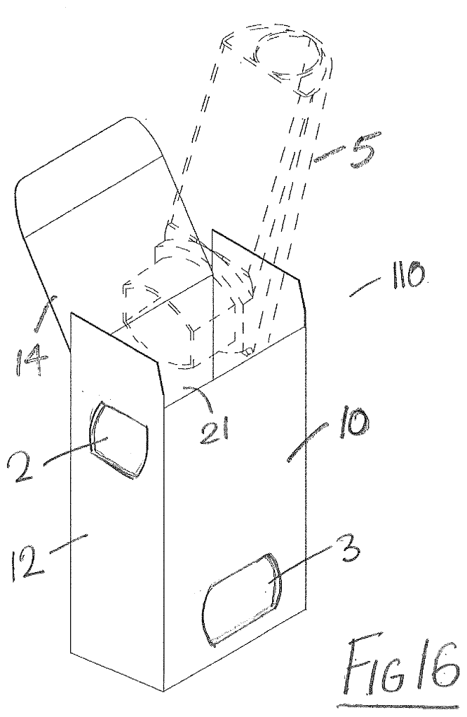

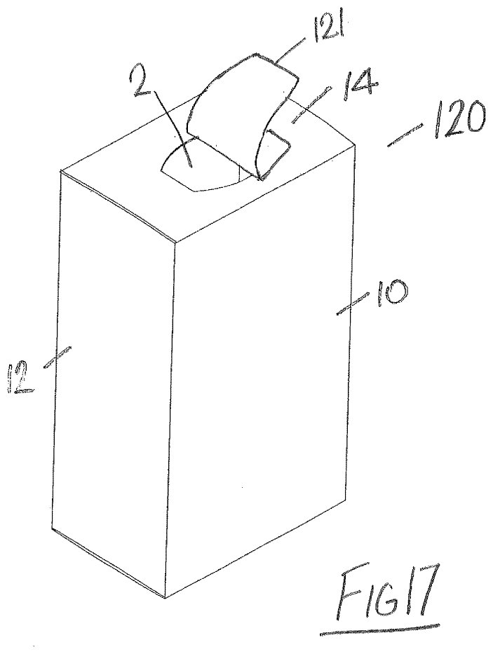

Referring now to FIG. 15, there is shown a further metered dose inhaler packaging carton according to a sixth embodiment of the invention, indicated generally by the reference numeral 100. Parts similar to those described previously are assigned the same reference numerals. In this case, the inlet opening 2 and the outlet opening 3 are provided spaced-apart at a top of the rear panel 11 and at a bottom of the front panel 10 respectively, at an opposite side of the metered dose inhaler packaging carton 100. Referring now to FIG. 16, there is shown a further metered dose inhaler packaging carton according to a seventh embodiment of the invention, indicated generally by the reference numeral 110. Parts similar to those described previously are assigned the same reference numerals. In this case, the inlet opening 2 is provided at a top of the side panel 12 and the outlet opening 3 is provided at a bottom of the adjacent front panel 10.

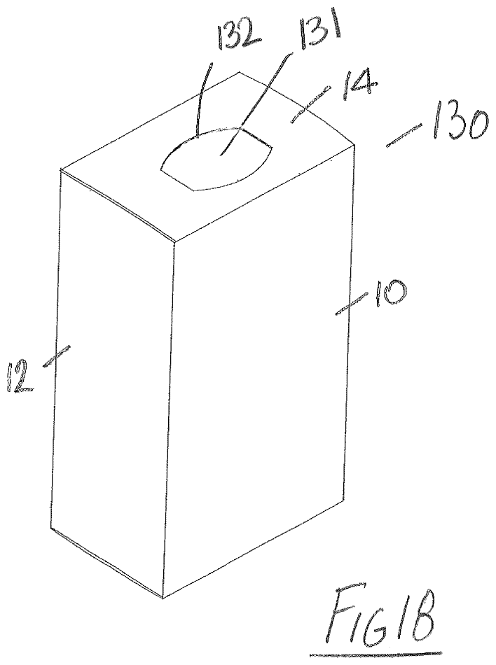

Referring now to FIG. 17, there is shown a further metered dose inhaler packaging carton according to an eighth embodiment of the invention, indicated generally by the reference numeral 120. Parts similar to those described previously are assigned the same reference numerals. In this case, a peel-away closure flap 121 is initially mounted across the inlet opening 2 to initially close the inlet opening 2. The peel-away flap 121 may be discarded after initial removal, or it may be a peel and re-seal flap 121. A similar flap 121 may be provided at the outlet opening 3 (not shown).

Referring now to FIG. 18, there is shown another metered dose inhaler packaging carton according to a ninth embodiment of the invention, indicated generally by the reference numeral 130. Parts similar to those described previously are assigned the same reference numerals. In this case, each of the inlet opening 2 and the outlet opening 3 are closed initially by a removable closure flap 131 defined by lines of weakness 132 stamped or otherwise formed in the top panel 14 and bottom panel 24, the line of weakness 132 extending partially through the material of the top panel 14 and bottom panel 24 in the shape of the inlet opening 2 and outlet opening 3. Prior to use of the packaging carton 130 as a spacer, the removable closure flap 131 is simply pushed out of the top panel 14 and bottom panel 24 to reveal the inlet opening 2 and outlet opening 3. The line of weakness 132 may alternatively be formed by perforations which allow the user to push out the closure flap 131 prior to use.

The storage container may be provided in any suitable carton style (with additional or different tabs, flaps, tuck-ins, shapes, etc.) or material or moulding, that uses a primary pack or carry pack concept with two holes and can act as a portable spacer.

The terms “comprise” and “include”, and any variations thereof required for grammatical reasons, are to be considered as interchangeable and accorded the widest possible interpretation.

The invention is not limited to the embodiments hereinbefore described but may be varied in both construction and detail within the scope of the appended claims.

1. A metered dose inhaler storage container configured to form a spacer for a metered dose inhaler, the storage container having a pair of spaced-apart openings in a side wall of the storage container comprising an inlet opening for reception of an outlet of the metered dose inhaler and an associated outlet opening for engagement by a mouth of a user to inhale medication discharged from the metered dose inhaler and delivered through the storage container.

2. The storage container as claimed in claim 1, wherein the storage container comprises a packaging carton or box for reception and storage of the associated metered dose inhaler prior to use and in between uses of the metered dose inhaler by a patient.

3. The storage container as claimed in claim 1, wherein a removable closure is provided at each opening.

4. The storage container as claimed in claim 3, wherein the removable closure is a flap mounted across the opening.

5. The storage container as claimed in claim 4, wherein the flap is a tear-away flap.

6. The storage container as claimed in claim 4, wherein the flap is mounted in the opening.

7. The storage container as claimed in claim 6, wherein a periphery of the flap connects to a periphery of the opening by a perforated seam.

8. The storage container as claimed in claim 4, wherein the flap is hingedly mounted at a side of the opening for movement between a closed position extending across the opening and an open position away from the opening.

9. The storage container as claimed in claim 3, wherein the removable closure is a peel-away tab.

10. The storage container as claimed in claim 9, wherein the tab is a peel and re-seal tab.

11. The storage container as claimed in claim 1, wherein the inlet opening is shaped for complementary reception and engagement of the mouthpiece of the metered dose inhaler within the inlet opening.

12. The storage container as claimed in claim 1, wherein the storage container is impregnated with an anti-microbial agent.

13. The storage container as claimed in claim 12, wherein the anti-microbial agent comprises anti-microbial metal ions.

14. The storage container as claimed in claim 1, wherein at least an interior surface of the storage container is coated with an anti-static material to minimise drug retention.

15. The storage container as claimed in claim 1, wherein the storage container has a coating of plastics material.

16. The storage container as claimed in claim 1, in combination with an associated metered dose inhaler which houses within the storage container.

17. The combination as claimed in claim 16, wherein the opening in the top panel and/or the bottom panel has a periphery shaped to receive and engage an exterior of the outlet of the metered dose inhaler.

18. A blank for forming the metered dose inhaler storage container as claimed in claim 1.

19. The blank as claimed in claim 18, wherein the blank comprises a front panel, rear panel and side panels arranged side by side in a row joined along side edges, the front panel being connected at one side edge to a first side panel by a first fold line and on the other side edge to one side edge of a second side panel by a second fold line, an opposite side edge of the second side panel connecting to a first side edge of the rear panel by a third fold line, a connector flap projecting outwardly of a second side edge of the rear panel by a fourth fold line, an adhesive strip on a face of the connector flap for engagement with the first side panel, a top panel hingedly connected to a top edge of the rear panel and a bottom closure flap connected to a bottom edge of the rear panel, a bottom panel connected to a bottom edge of the front panel and a top closure flap connected to a top edge of the front panel.

20. The blank as claimed in claim 19, wherein the panels are of rectangular shape.
